(19) United States
(12) Patent Application Publication
(10) Pub. No.: US 2009/0005812 A1
(43) Pub. Date: Jan. 1, 2009

(54) Portable chiropractic adjustor

(75) Inventor: Arlan W. Fuhr, Phoenix, AZ (US)

Correspondence Address: GREENBERG TRAURIG (PHX), INTELLECTUAL PROPERTY DEPARTMENT, 2450 COLORADO AVENUE , SUITE 400E, SANTA MONICA, CA, 90404, US

(73) Assignee: Activator Methods International, Ltd., Phoenix, AZ (US)

(21) Appl. No.: 12/157,540

(22) Filed: Jun. 11, 2008

Related U.S. Application Data
(63) Continuation of application No. 11/159,546, filed on Jun. 23, 2005, now abandoned.

(51) Int. Cl. A61F 5/00

(52) U.S. Cl. 606/239; 606/237

(57) ABSTRACT

A portable power operated chiropractic adjustor, manipulator or thruster for applying an adjustment energy to a patient through a plunger having a resilient or cushioned head with the energy applied to the plunger being supplied by non-manual sources and the impulse is adjustable or tunable along with having annunciators or indicators for preload and readiness to operate. The adjustor also allows for settings of single or multiple strokes. The power source may be internal such as from a rechargeable battery, removable rechargeable battery pack, or air cartridge depending on whether it is electrically or pneumatically operated.

### FIELD OF INVENTION

The present invention relates generally to a portable chiropractic adjustor for use in chiropractic adjustment of musculoskeletal structures. More, particularly, this invention concerns an improved power operated chiropractic-adjusting device for use in spinal manipulative therapy to apply impact forces or thrusts to a human body.

### DESCRIPTION OF THE PRIOR ART

The chiropractic art is generally concerned with adjusting misaligned body structures by manually manipulating the various joints in the human body. Of more specific interest in the art, however, is the spinal column which is comprised of several interconnected musculoskeletal structures or vertebrae. Unlike other, less critical body structures, the spinal column must be treated or manipulated with extreme caution because of its link with the central nervous system.

The human spine is susceptible to many different pathologic abnormalities including misalignment, miscellaneous trauma and pain, and degeneration as a result of age or disease. By employing various physical therapy techniques, though, a chiropractor, or one skilled in the chiropractic art, may be able to successfully treat a pathologic spine. Successful treatment will not only relieve any pain or discomfort that the patient might be suffering, but will also improve the overall quality of life of that patient.

One common spinal-adjustment technique involves applying thrusts or forces to the afflicted region of the spine. In particular, this technique involves either “mobilizing” the spine (i.e. passively moving the spine with relatively slow cyclic or oscillatory motion), or “manipulating” the spine (i.e. applying an impulsive thrust or force in a well-defined direction to a specific region of the spine). Depending on professional affiliations, this technique is referred to as chiropractic adjustment, osteopathic manipulation, orthopedic manual therapy, and/or spinal manipulative therapy.

There are several well-known procedures or techniques for “manipulating” or administering impulsive thrusts to a spine. One technique involves applying one or more rapid thumb thrusts to misaligned or afflicted vertebrae. Thumb thrusts, however, tend to be both imprecise in magnitude and location and tiresome to administer. Another technique involves using a manually operated chiropractic-adjusting instrument. For instance, U.S. Pat. No. 4,116,235, issued to Fuhr et al. (“Fuhr”); Fuhr U.S. Pat. No. 6,702,836; Fuhr U.S. Pat. No. 6,379,375; Keller et al. U.S. Pat. No. 5,626,615; Keller et al. U.S. Pat. No. 5,656,017; and U.S. Pat. No. 4,498,464, issued to Morgan, Jr., disclose such instruments.

Throughout the years it has also been known that power driven devices at times can offer benefits or advantages in use over the manually operated devices.

Electric solenoid operated adjustors such as ones described in Evans U.S. Pat. No. 4,841,955 issued in 1989 or Adelman U.S. Pat. No. 4,682,490, issued in 1987, can provide adjusting and controllability benefits over manual devices. However, using an electrical appliance close to the body can be potentially hazardous and even prohibited by governmental regulatory agency rules or regulations and power supply cords can get in the way.

Thus, numerous efforts have been made to develop a power operated thrusters with all of the desired features and benefits required for safe and varied usage of such devices. Examples of such an approach in pneumatic operated thrusters is shown in U.S. Pat. No. 4,716,890, issued in 1988 to Bichel and references cited in the Bichel patent.

While the Bichel thruster as described did seek to overcome disadvantages presented in prior art devices, it still did not provide certain features and advantages required to achieve wide spread acceptance and use by chiropractic practitioners.

By way of example, it may be noted that such prior devices including Bichel are capable of only delivering a single thrust or stroke, provide only manual adjustability of stroke lengths; provide force adjustment by changing stroke length and change air pressure only at the compressor or supply source. In addition they involve complicated multiple parts designs which make them more costly to manufacture and more difficult and costly to maintain or use. Recently, a pneumatic thruster of Frye U.S. Pat. No. 6,503,211 has solved some of the problems and objections of the field.

### OBJECTS AND SUMMARY OF THE INVENTION

The primary object of the present invention is to provide an improved power operated chiropractic instrument that is “tunable” or settable as to load, amplitude, and frequency within a user selected range of natural frequency.

A more specific object of the present invention is to provide a portable device with annunciators or indicators of settings such as preload and readiness to operate.

An even more specific object is to provide self contained power source for the adjusting instrument which can be rechargeable or replaceable.

These together with other objects and advantages which will become subsequently apparent reside in the details of construction and operation as more fully hereinafter described and claimed, reference being had to the accompanying drawings forming a part hereof, wherein like numerals refer to like parts throughout.

The foregoing is considered as illustrative only of the principles of the invention. Further, since numerous modifications and changes will readily occur to those skilled in the art, it is not desired to limit the invention to the exact construction and operation shown and described, and, accordingly, all suitable modifications and equivalents may be resorted to, falling within the scope of the invention.

### BRIEF DESCRIPTION OF THE DRAWINGS

FIG. 1 is a perspective view of the chiropractic adjustor of the present invention.

FIG. 2 is a side plan view of the adjustor with the cover removed and illustrating the structure and relationship of the components.

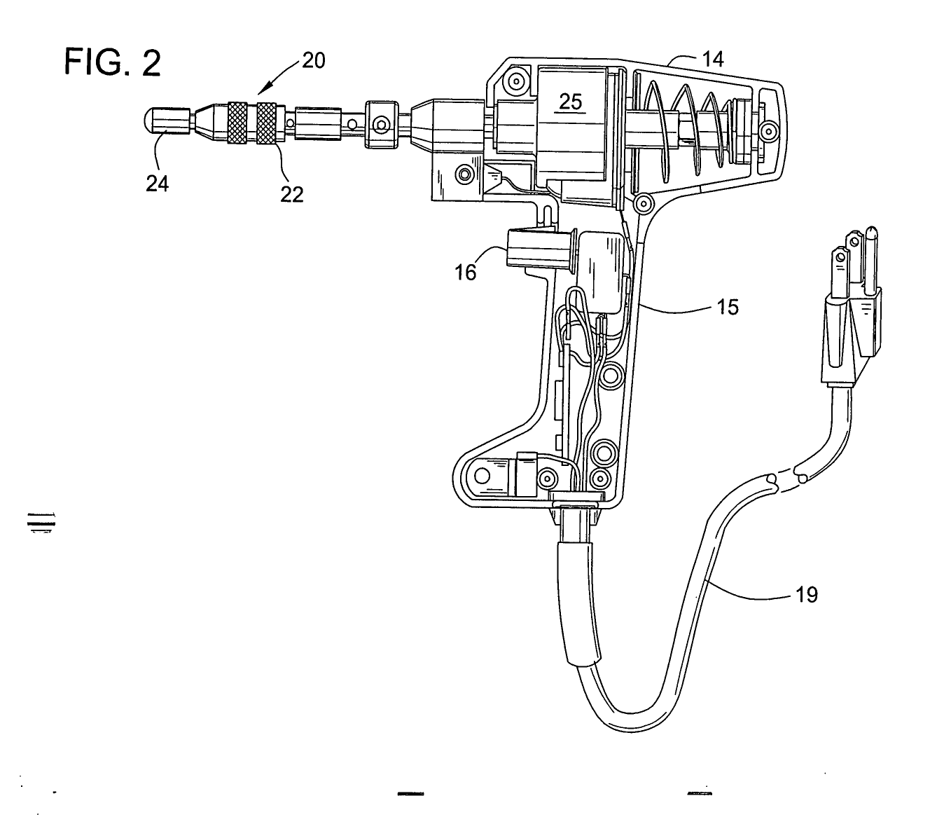

FIG. 3 is a perspective view of an alternative form of the adjustor; and

FIG. 4 is another alternative form of adjustor shown in perspective.

### DETAILED DESCRIPTION OF PREFERRED EMBODIMENT

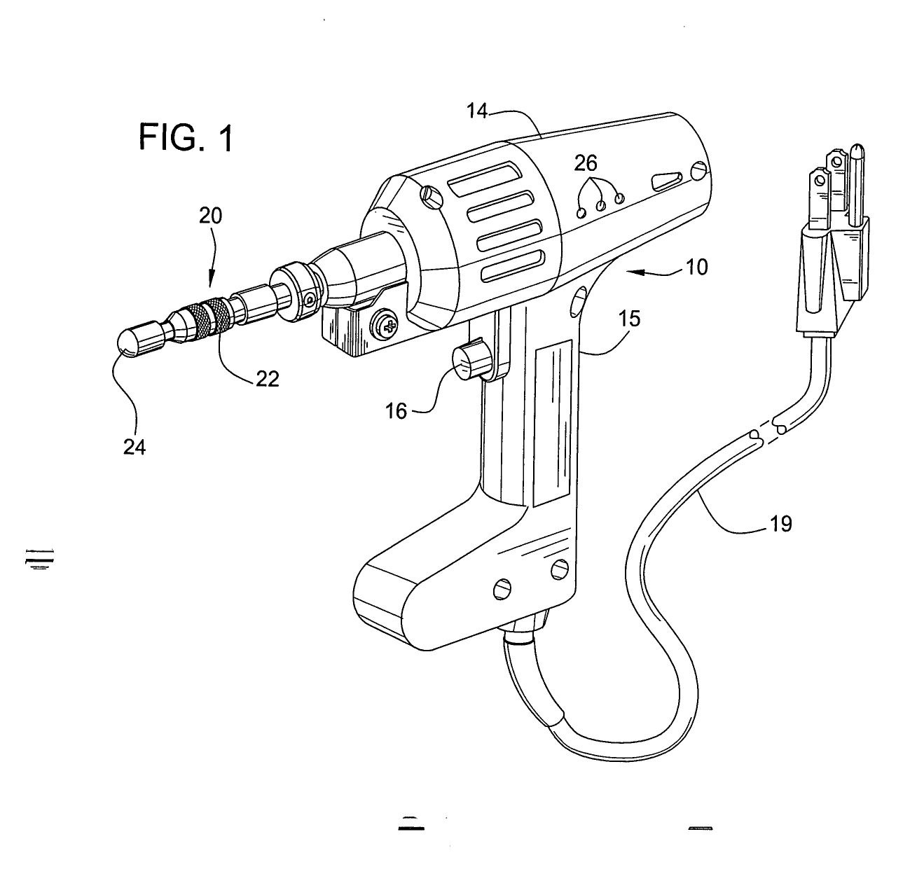

Referring to FIG. 1, the present power operated chiropractic adjustor 10 may take on the form of a pneumatic adjustor similar to U.S. Pat. No. 6,503,211 or an electric adjustor similar to U.S. Pat. No. 4,841,955. In either event, there is a generally cylindrical housing portion 14 for the operating thrusting mechanism and a handle or grip 15 portion which can contain the power input source and a finger operated trigger 16. Where the input power is an outside air line (not shown) or an electric cord 19 the grip portion can contain necessary valves or contact connections, respectively.

The forward or head end 20 of the instrument has a rigid plunger or rod 22 that may be removeably connected and carries the cushioned head on tip 24. The plunger can be interchangeable with various single or dual style forms.

A preload device, such as described in U.S. Pat. No. 6,702,836 can be preferably incorporated in the head end.

The grip portion can also be used for replaceable or rechargeable power input sources. In the case of a pneumatic device disposable or even rechargeable cartridges can be employed so that there is no feed line required.

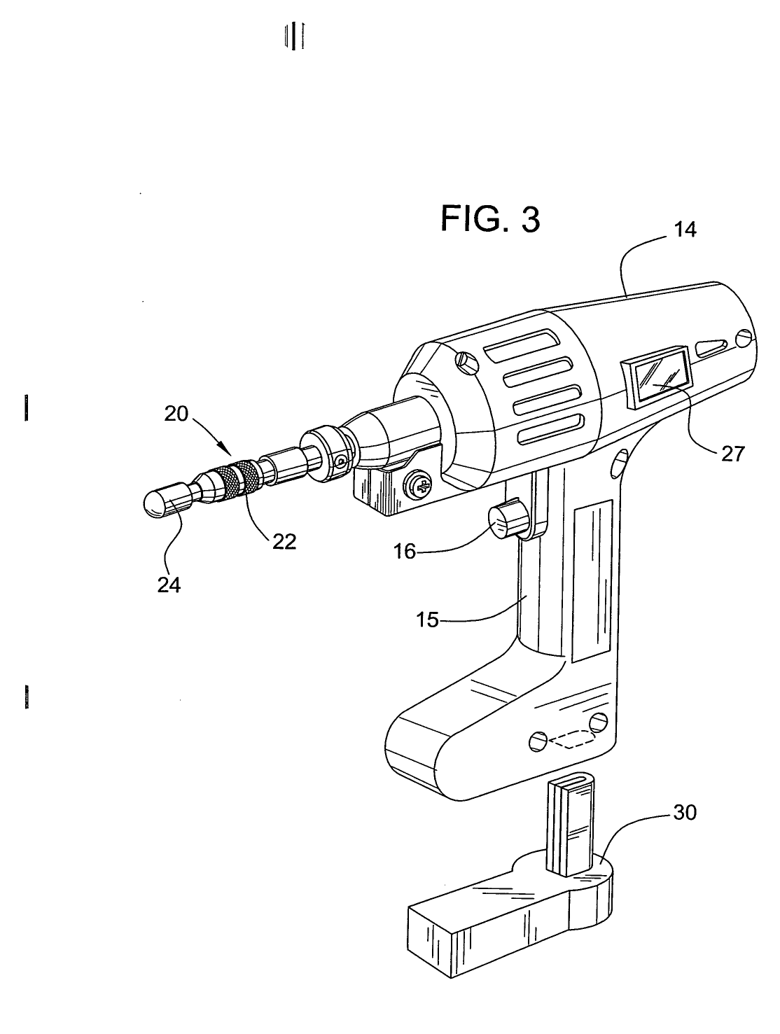

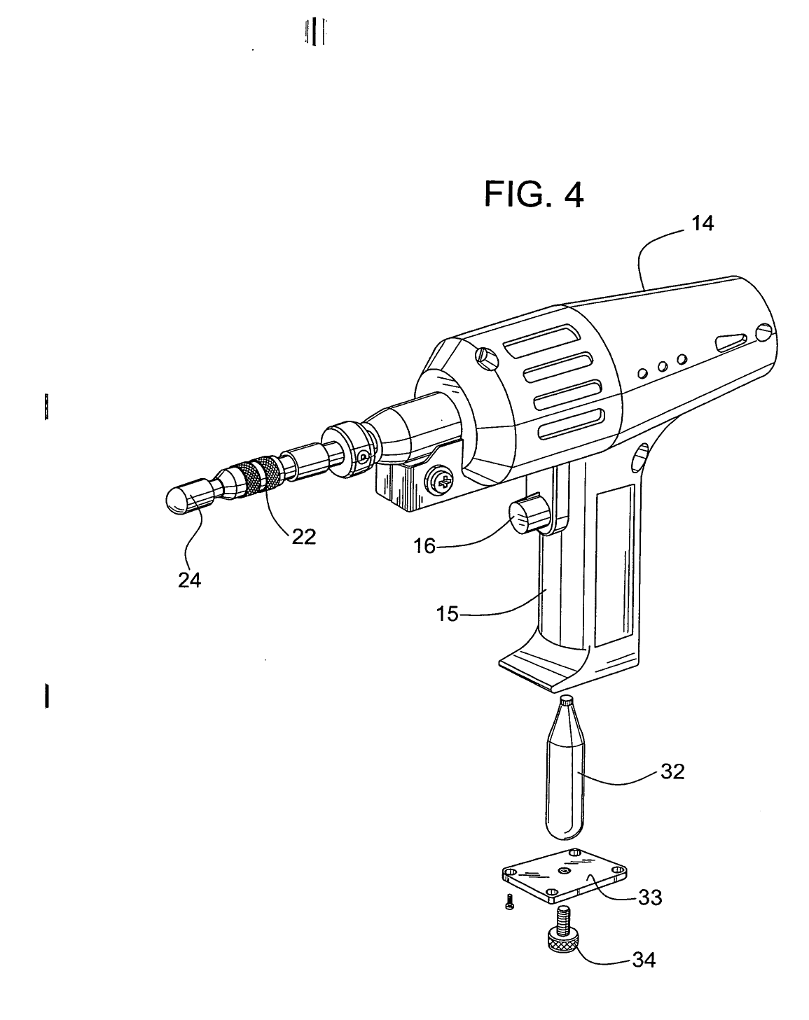

For an electric operated instrument a rechargeable battery 30 (FIG. 3) can be mounted in the grip portion along with appropriate circuitry for control power to activate a solenoid device and even a self contained recharger. Alternatively, a removal battery pack 30 can be used that would be charged in a free standing charger and replaceable with a spare charged battery pack when one pack is being charged. As shown in FIG. 4, a removable air or gas cartridge 32 can be used for portability. A plate 33 and thumb screw 34 holds the cartridge in place.

In accordance with one of the important features of the present invention, the instrument is tunable. In the case of a pneumatic instrument valve adjustments may be included for controlling the amount of thrust as well as single and multiple thrusts. U.S. Pat. No. 6,503,211 may be referenced for such capabilities.

In an electric version, rheostat or push button controls can be used. Alternatively a trigger circuit that includes conventional electronic components can deliver selective power in a single pulse or multiple pulses to drive a solenoid type device 25 for generating the thrust. Such controls are generally known and available, for example, in handheld, portable battery operated tools.

The circuitry can also contain microprocessor devices for data storage such as number and duration of thrusts, for example, and the data collected can be down loadable to a computer loaded with diagnostic software and even to maintain a patent database.

Another important aspect of the present invention is that the instrument is provided with annunciation such as LED lights 26, or an LCD display 27 (FIG. 3), or possibly even an audio annunciator to provide feedback to the user as to such things as preload settings, loading status, readiness, force settings, and even values of impulse frequency or amplitude. LED lights 26 could be red to yellow to green, for example, for preload status. LCD display 27 could be graphic, alpha numeric or pictorial.

1. A portable power operated chiropractic adjustor for applying an adjustment energy to a patient through a plunger having a cushioned head, comprising, a body portion containing powered thrust delivering means, a grip means including a trigger to activate said thrust delivery means, tunable control means for selectively applying a preselected amount of force to a patient, and annunciator means for indicating setting and status of the instrument.

2. A portable power operated chiropractic adjustor as claimed in claim 1, wherein said thrust delivery means includes preload means and said annunciator means indicates preload status.

3. A portable power operates chiropractic adjustor as claimed in claim 1 or 2, wherein the annunciator means includes LED lights.

4. A portable power operated chiropractic adjustor as claimed in claim 1, wherein said annunciator means includes an LCD display.

5. A portable power operated chiropractic adjustor as claimed in claim 1, wherein said annunciator means includes an audio output.

6. A portable power operated chiropractic adjustor as claimed in claim 1, wherein said powered thrust delivery system is electrical.

7. A portable power operated chiropractic adjustor as claimed in claim 1, wherein said electrical power source is a battery.

8. A portable power operated chiropractic adjustor as claimed in claim 7, wherein said battery is rechargeable.

9. A portable power operated chiropractic adjustor as claimed in claim 7, wherein said battery is a removable and rechargeable pack.

10. A portable power operated chiropractic adjustor as claimed in claim 1, wherein said powered thrust delivery system is pneumatic.

11. A portable power operated chiropractic adjustor as claimed in claim 10, wherein said pneumatic power source is an air cartridge.
